(19) United States
(12) Patent Application Publication
(10) Pub. No.: US 2012/0182523 A1
(43) Pub. Date: Jul. 19, 2012

(54) METHODS FOR REDUCING TEST-RETEST VARIABILITY IN TESTS OF VISUAL FIELDS

(75) Inventor: Harry J. Wyatt, New York, NY (US)

(73) Assignee: THE RESEARCH FOUNDATION OF STATE UNIVERSITY OF NEW YORK, Albany, NY (US)

(21) Appl. No.: 13/499,110

(22) PCT Filed: Sep. 30, 2010
(86) PCT No.: PCT/US10/50856

§ 371 Date: Mar. 29, 2012

Related U.S. Application Data
(60) Provisional application No. 61/247,085, filed on Sep. 30, 2009.

(51) Int. Cl. A61B 3/024

(52) U.S. Cl. 351/224; 351/246

(57) ABSTRACT

The invention relates to test-retest variability in tests of visual fields, and methods and systems useful in reducing this variability. The various methods and systems of the invention use gaze-direction data to improve the estimate of scotoma edges and to otherwise adjust for test-retest variability in perimetry. This may be useful in assessing progression of a patient's condition, such as glaucoma

### GOVERNMENT RIGHTS

This invention was made with government support under Grant Nos. 5T35EY00707917 and 5R03EY01454903 awarded by the National Eye Institute of the National Institutes of Health. The government has certain rights in the invention.

### FIELD OF THE INVENTION

The invention relates to test-retest variability in tests of visual fields, and methods to reduce this variability.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The principal clinical method for functional assessment of patients' visual fields is perimetry; in particular, computerized perimetry or “standard automated perimetry” (SAP). By far the most common type of perimeter used is the “static” perimeter, in which small, brief flashes of light are presented at a number of different locations, while a patient looks steadily at a fixation target. In other words, while looking at one target that is continuously visible, the patient tries to be aware of the brief appearance of a target at a different location. When the patient notices such a flash of light, he or she presses a button. This is not a trivially easy task, but it is regarded as the principal way in which visual function away from the point of gaze can be tested. Although in the normal visual field, test-retest variability in this context is relatively low (on the order of 1-2 dB), in damaged visual fields it can become much higher. This makes it difficult to determine whether a patient's condition is stable or progressing, since the determination amounts to assessing the presence or absence of a trend (progression) in the presence of noise (test-retest variability).

A “defect” means reduced sensitivity to visual stimuli in some part of the visual field. A number of pathologies, either of the eye or of the later visual pathway, result in defects of the visual field. One important example is glaucoma, which affects the retina and the optic nerve of an eye.

Aside from actual progression, the possible causes of increased test-retest variability in damaged fields include (i) increased variability of retinal ganglion cell (RGC) behavior, due to damage, (ii) reduced numbers of RGCs and/or reduced regularity of the RGC array, also due to damage, and (iii) fixational eye movements during testing.

Furthermore, one of the difficulties in assessing details of scotomas, and one which may be related to variability, is the question of where scotoma edges are located. To date, in experimental work studying sensitivity near edges of scotomas or the edge of the blindspot (using it as a physiological scotoma), the location of the edge relative to the test locations has generally been unknown. To clarify this, in a commonly encountered situation, a row of three test locations gives the following results: at the first location, sensitivity is repeatedly found to be near normal, while at the third location sensitivity is repeatedly found to be essentially absent. At the second (middle) test location, results may be like those of the first location, or like those of the third location, or be highly variable from test to test. In the case of high variability at the middle location, all that can be said about the boundary between healthy and damaged field is that it lies between the first and third locations, which—in the case of the usual 6 or 2 degree grids—locates the boundary to within 12 or 4 degrees, respectively. Examples of such situations are commonplace findings in repeated visual fields near edges.

One reason why scotoma edge location is an important issue is that sensitivity can change rapidly as one crosses such an edge. If a visual field test location lies near to an edge, then, a small eye movement might make a large difference in sensitivity at the location to which a test stimulus is delivered. Moreover, if fixational eye movements are involved in generating test-retest variability, studying eye movements during visual field testing might illuminate the issue. However, there have heretofore been no published studies of sensitivity near edges in which the time course of eye movements has been related to the time course of the test results (i.e., the time course of the staircase of test presentations used to arrive at the test results).

If the gaze direction (fixation error) were known for each test flash in a perimetric determination, that would mean that the actual retinal location of each test flash would be known. It seemed possible that in such a situation the spatial distribution of test flash contrasts and subject responses could be used to determine the most likely boundary between healthy and damaged field. If the spatial pattern of damage is relatively simple, with substantial areas of healthy and damaged field separated by simple boundaries, then a manageable number of repeat tests might provide enough data to estimate the retinal location of the boundary. In the inventor's work, visual sensitivity near the blind spot was measured in normal subjects and gaze direction was measured concurrently. A post hoc analysis indicated that a substantial part of the test-retest variability could be accounted for plausibly by the eye movements measured for each subject (Wyatt H J. IOVS 2010; 51:ARVO E-Abstract 5496).

From a clinical standpoint, a frustrating aspect of visual field testing with static perimetry is that there can be a lot of variation between one test and the repeat of the same test, either on the same day or at some later time. In a completely normal eye, this variation is not large, but in a damaged eye it can become very large. In fact, the variability becomes greatest in areas where there are defects in the visual field. This means that it can be difficult to tell whether a change in a test value is due to variability, or whether it is due to a real change of the underlying condition of the patient. For example, one of the difficult aspects of glaucoma is “progression,” namely, worsening of the disease. Test-retest variability makes assessment of progression very difficult. Thus, for at least these reasons, there is a need in the art for novel methods of reducing test-retest variability in visual fields.

### SUMMARY OF THE INVENTION

In an embodiment, the invention includes a method of improving or at least partially correcting test-retest variability in perimetry, comprising: recording the parameters of gaze direction, time, flash brightness and subject response to a series of test flashes at a series of locations to generate visual field results; and adjusting test-retest variability in the visual field results by accounting for gaze direction. The step of adjusting test-retest variability may comprise associating the gaze direction with the flash brightness and the patient response for each said time at a said location; constructing a spatial map of patient responses for each of a series of the said locations; and accounting for test-retest variability based upon the spatial maps.

In another embodiment, the invention includes a method of gauging a progression of a patient's condition, comprising: in a first session, recording the parameters of gaze direction, time, flash brightness and subject response to a series of test flashes at a series of locations to generate a first set of visual field results; in at least one additional session, recording the parameters of gaze direction, time, flash brightness and subject response to a series of test flashes at a series of locations to generate an additional set of visual field results for each of the at least one additional session; and either (i) adjusting test-retest variability in the first set of visual field results and the at least one additional set of visual field results by accounting for gaze direction, and gauging the progression based on a change in the adjusted first set of visual field results as compared with the adjusted at least one additional set of visual field results; or (ii) gauging the progression based on a comparison of the parameters recorded in the first session with the parameters recorded in the at least one additional session. The step of adjusting test-retest variability in the first set of visual field results and the at least one additional set of visual field results may comprise: associating the gaze direction with the flash brightness and the patient response for each said time at a said location in each of the first set of visual field results and each of the at least one additional set of visual field results; constructing a spatial map of patient responses for each of a series of the said locations; and accounting for test-retest variability based upon the spatial maps. The patient's condition may be glaucoma, other diseases of the eye, or other clinical or disease conditions featuring a deleterious progression in pathology of the eye.

In another embodiment, the invention includes a perimetry system, comprising: a component that interfaces with a patient's eyes to enable performance of visual field testing; and a computer in electronic communication with the component, and configured to: record the parameters of gaze direction, time, flash brightness and subject response to a series of test flashes at a series of locations to generate visual field results during the visual field testing, adjust test-retest variability in the visual field results by accounting for gaze direction, and generate an output of results to a user of the perimetry system. The computer may be further configured to: associate the gaze direction with the flash brightness and the patient response for each said time at a said location; construct a spatial map of patient responses for each of a series of the said locations; and account for test-retest variability based upon the spatial maps. The computer may be further configured to: store the recorded parameters from successive test sessions of the patient; and gauge a progression of the patient's condition based on a change in either the recorded parameters from the successive test sessions, or a change in the adjusted visual field results from the successive test sessions. The patient's condition may be glaucoma, other diseases of the eye, or other clinical or disease conditions featuring a deleterious progression in pathology of the eye.

In another embodiment, the invention includes a computer readable medium having computer executable components for: recording the parameters of gaze direction, time, flash brightness and subject response to a series of test flashes at a series of locations to generate visual field results during perimetry, adjusting test-retest variability in the visual field results by accounting for gaze direction, and generating an output of results. The computer readable medium may further comprise components for: associating the gaze direction with the flash brightness and the patient response for each said time at a said location; constructing a spatial map of patient responses for each of a series of the said locations; and accounting for test-retest variability based upon the spatial maps. The computer readable medium may further comprise components for: storing the recorded parameters from successive test sessions of the patient; and gauging a progression of the patient's condition based on a change in either the recorded parameters from the successive test sessions, or a change in the adjusted visual field results from the successive test sessions. The patient's condition may be glaucoma, other diseases of the eye, or other clinical or disease conditions featuring a deleterious progression in pathology of the eye.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts, in accordance with an embodiment herein, a chart demonstrating how changes of gaze direction can generate test-retest variability.

FIG. 2 depicts, in accordance with an embodiment herein, an illustrative set of test results for a single nominal test location, in which (a) depicts test results for that location in a single graph; (b) depicts a series of nine plots from the same data, but generated after separating the results according to gaze direction; and (c) depicts data converted to a smoothed contour plot, where the separation by gaze direction creates a “microfield” in the vicinity of the nominal test location. Note that data depicted in this FIG. 2 are only for purposes of illustration, and are not based on actual human testing.

FIG. 3 depicts, in accordance with an embodiment herein, a test array used for right eyes studied. The diamond represents the fixation target; the dark oval is an average blind spot in position and size.

FIG. 4 depicts, in accordance with an embodiment herein, average sensitivity and variability for one subject (S6) shown as grayscale contour plots. Star indicates the location used in subsequent analysis. Sensitivity and variability varied from 0.0 to 16.3 dB and 0.0 to 6.9 dB, respectively.

FIG. 5 depicts, in accordance with an embodiment herein, post hoc data analysis for the subject of FIG. 4 and the test location indicated by the star in FIG. 4: (13 deg, −3 deg) relative to fixation. Open (filled) symbols represent stimuli that were seen (not seen). At left, all data are shown at one location; at right, data are plotted at the retinal location determined by looking up the gaze direction for each flash. The fitted functions (gray lines) that maximized likelihood were sensitivity=−12.6 dB (ignoring eye position) and a step function from −3.0 dB relative to normal to about −19 dB, positioned very near to the nominal test location. The steepness of the probability distribution was beta=0.50 (left) and beta=2.64 (right), amounting to an 81% reduction in variability when gaze direction was considered. Note that, in the data depicted in this FIG. 5, only gaze-direction data for horizontal changes of gaze were available, and thus the test location for which the post hoc analysis was carried out (the starred location) was selected where the sensitivity edge appeared to run nearly vertically.

FIG. 6 depicts, in accordance with an embodiment herein, fits for a subject (S7) with 2-dimensional gaze-direction data. Four high-variability locations were analyzed. Sensitivity varied from 0.0 to 15.9 dB.

FIG. 7 depicts, in accordance with an embodiment herein, a perimetry system for functional assessment of patients' visual fields, configured with improved or at least partially corrected test-retest variability features.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described.

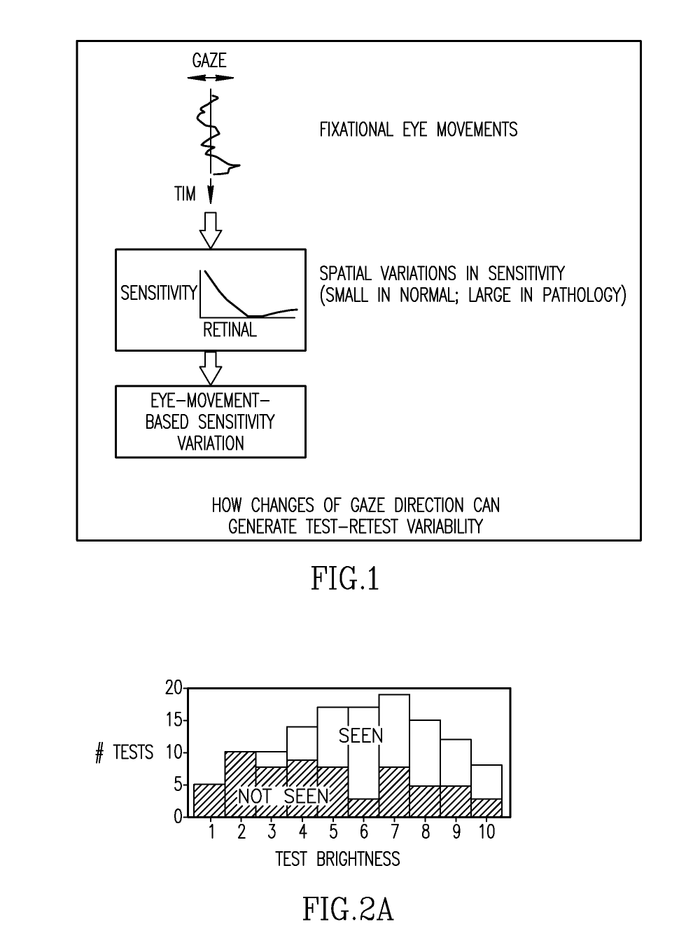

The cause of test-retest variability in perimetry in not an entirely settled issue, and there are several views of the matter. For the case of retinal or optic nerve damage, one view is that nerve cells in the retina become more variable in their behavior when they are damaged. Another view is that retinal damage causes loss of some of the cells, so that the retina becomes patchy on a very fine scale, showing up as variability of test results. However, these postulates are not needed to explain test-retest variability in damaged visual fields; instead, variability can result from ordinary behavior on the part of patients. When people try to look steadily at a target, especially if they are trying to pay attention to the rest of their visual field at the same time, their eyes continually move around by small amounts. The amount of movement varies from one person to another; typically, the range of such movement might be 0.2 degrees of arc for one person and as much as 0.6 or 1.0 degrees for another. As the gaze direction moves around, the location where any given test flash falls on the retina also moves around. For a healthy eye, this won't have any noticeable effect on the results of a visual field test. However, when a visual field has been damaged, visual sensitivity can change drastically over short distances. This creates a mechanism for test-retest variability that does not require any hypotheses beyond what is already known; that is, (i) sensitivity can change rapidly over short distances in a damaged field, and (ii) patients' eyes move around during testing. The concept is shown in FIG. 1.

There are different ways in which reduction of test-retest variability due to changes of gaze direction may be accomplished. One is to “stabilize” the stimulus; this refers to putting the stimulus at a single retinal location in spite of the shifts of gaze direction. This is possible, but it is not simple. For example, the so-called “microperimeters” stabilize stimuli, but they are complex and expensive. An alternative approach would be to record gaze direction during perimetry and then adjust the visual field results after the test is completed. Although this sounds complicated and costly, some of the commercial perimeters (e.g., the Humphrey Field Analyzer made by Zeiss Meditech) already incorporate a video camera aimed at the patient's eye. Some perimeters, therefore, already contain the necessary hardware to effectuate this technique, even though the devices aren't currently configured to accomplish it.

Two types of data are required to do this: (i) a record of gaze direction during a given visual field test, and (ii) a record of the times and brightnesses of each test flash delivered along with the patient's responses (“seen” if the button was pressed, “not seen” otherwise).

The post-test analysis may then proceed as follows: a) For each test flash location, look up the history of flash brightnesses and patient responses. This will provide a sequence of times (relative to the start of the test), brightnesses, and responses. b) For each time at a given location, look up the gaze direction and associate it with each test brightness and patient response. c) For a given location, construct a spatial map of the seen and not seen brightnesses. In the case of an ideal patient, where all of the variability is due to gaze shifts, this will produce a map where the true nature of the map is apparent.

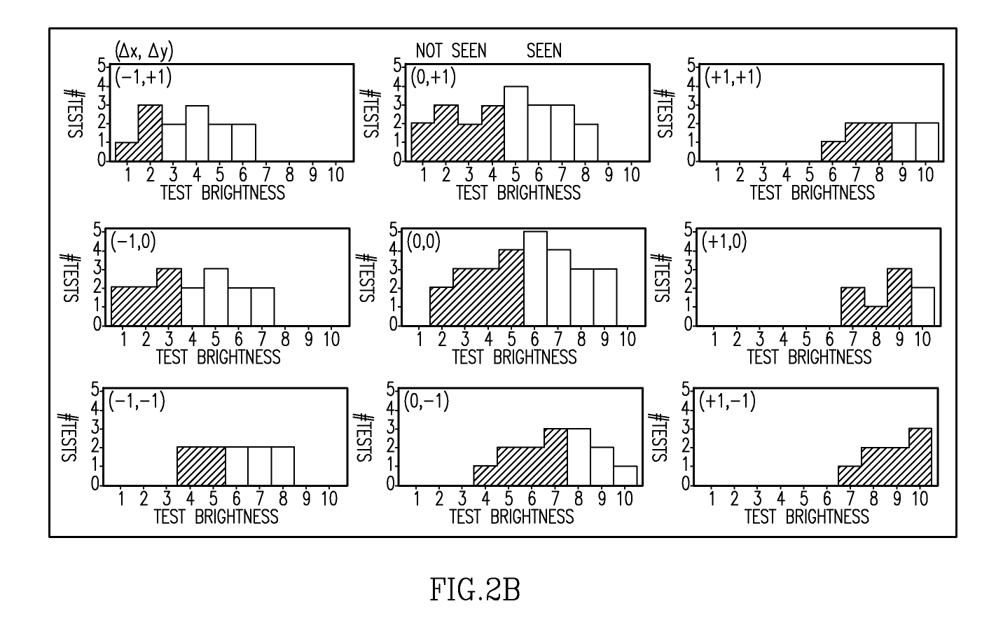

Depicted in FIG. 2 herein is a set of test results for a single nominal test location. All test results for that location are shown in a single graph at FIG. 2 (a). Note that for many test brightnesses, the flash is sometimes seen and sometimes not seen. This large amount of seen/not-seen overlap would correspond to high variability calculated for sensitivity at the test location in the usual manner. However, after separating the results according to gaze direction, the following set of plots depicted in FIG. 2 (b) might result from the same data. The nine plots correspond to different gaze-direction bins; these bins including the one in the center at the nominal location might be 0.25 deg×0.25 deg or 0.5 deg×0.5 deg, depending on how much the patient's gaze shifts.

These data can be converted to approximate thresholds for each bin, as shown in FIG. 2(c). If this is converted to a smoothed contour plot, it would look like the one shown in FIG. 2(c). Essentially, the separation by gaze direction creates a “microfield” in the vicinity of the nominal test location. For the idealized case shown, the separate plots show zero overlap of seen and not seen; that would mean that all of the test-retest variability was removed by taking gaze direction into account. Although real data will generally be noisier, at least some reduction is likely in most cases, since there is considerable evidence that changes in gaze direction cause a significant amount of test-retest variability in damaged visual fields. Testing shows evidence of considerable benefits from applying this approach, and the examples shown below provide further evidence of the functionality of this approach.

Therefore, in one embodiment, the present invention provides a method of improving or at least partially correcting test-retest variability in standard perimetry by recording gaze direction during perimetry and then adjusting the visual field results after the test is completed. In another embodiment, the present invention provides a method of at least partially correcting test-retest variability by recording gaze direction during a given visual field test, recording the times and brightnesses of some, most or all test flashes delivered along with corresponding some, most or all of the patient's responses, and then performing post-test analysis to at least partially correct test-retest variability. In another embodiment, post-test analysis includes the following procedure: a) look up history of flash brightnesses and patient response for each test flash location; b) look up the gaze direction and associate it with each test brightness and patient response for each time at a given location; and c) for a given location, construct a spatial map of the seen and not seen brightnesses.

In yet another embodiment, raw data (in the form of gaze direction, and times, brightnesses and patient response to test flashes) and/or corrected post hoc results from successive patient test sessions is combined. This may be especially useful in embodiments of the invention where an assessment is being performed as to whether or not a patient's condition has progressed. As noted above, “progression” relates to a change (and generally, a worsening) in condition over time, so having a time sequence of findings, if not raw data, may be important. In such embodiments, the invention thus includes a method of gauging the progression of a patient's condition, comprising combining raw data and/or corrected post hoc results from successive patient test sessions, and gauging the progression based on a change in the raw data and/or corrected post hoc results. This may be especially useful, for instance, in prognosing the progression of glaucoma, other diseases of the eye, or other clinical or disease conditions featuring a deleterious progression in pathology of the eye, as will be readily appreciated by those of skill in the art.

In another embodiment, the present invention provides a perimetry system for functional assessment of patients' visual fields, configured with improved or at least partially corrected test-retest variability features. The system, which in one embodiment includes a computer, may be configured to record gaze direction during perimetry and then adjust the visual field results after the test is completed. To accomplish this, the system may include components that allow for the recording of gaze direction during a given visual field test, and the recording of the times and brightnesses of some, most or all test flashes delivered along with corresponding some, most or all of the patient's responses. The system may be further configured with components capable of performing post-test analysis to at least partially correct test-retest variability, by: a) looking up history of flash brightnesses and patient response for each test flash location; b) looking up the gaze direction and associate it with each test brightness and patient response for each time at a given location; c) for a given location, constructing a spatial map of the seen and not seen brightnesses; and d) computationally improving or at least partially correcting for test-retest variability. In yet another embodiment, raw data (in the form of gaze direction, and times, brightnesses and patient response to test flashes) and/or corrected post hoc results from successive patient test sessions may be combined and analyzed by the system to render an assessment as to whether or not a patient's condition has progressed.

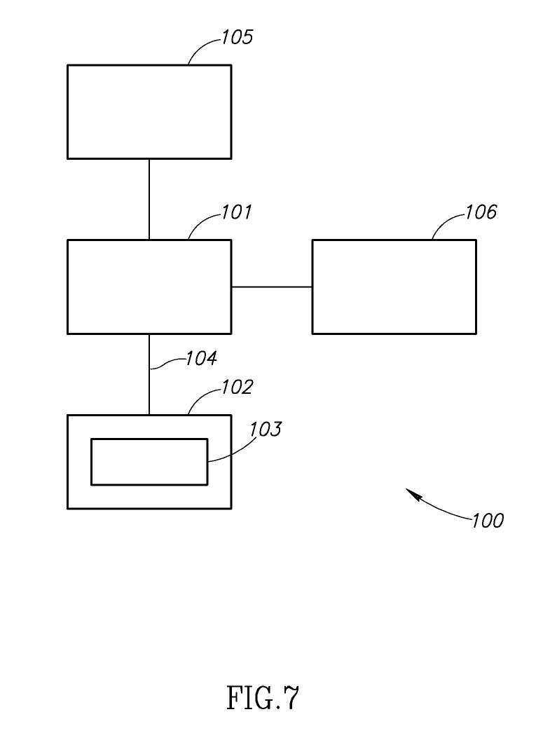

With reference to FIG. 7, the aforementioned system 100 may include a programmable central processing unit (CPU) 101 which may be implemented by any known technology, such as a microprocessor, microcontroller, application-specific integrated circuit (ASIC), digital signal processor (DSP), or the like. The CPU 101 may be integrated into an electrical circuit, such as a conventional circuit board, that supplies power to the CPU 101. The CPU 101 may include internal memory or memory may be coupled thereto 102. The memory 102 is a computer readable medium that includes instructions 103 or computer executable components that are executed by and control operation of the CPU 101. The memory 102 may be coupled to the CPU 101 by an internal bus 104. The memory 102 may comprise random access memory (RAM) and read-only memory (ROM). The memory 102 may also include a basic input/output system (BIOS), which contains the basic routines that help transfer information between elements within the system 100. The present invention is not limited by the specific hardware component(s) used to implement the CPU or memory components of the system. The system may also include an external device interface 105 permitting the user or a medical or other healthcare professional to enter control commands, such as a command to initiate visual field testing, a command to adjust one or more parameters of the testing, commands providing new instructions to be executed by the CPU, and the like, into the system 100. The system 100 may also include a component 106 that interfaces with a patient's eyes to enable performance of visual field testing. The various components of the system 100 may be coupled together by internal buses, each of which may be constructed using a data bus, control bus, power bus, I/O bus, and the like. The system 100 may include instructions executable by the CPU 101 for processing and/or analyzing the record of gaze direction, times, brightnesses and/or patient responses, and for providing a result to the user in which test-retest variability is improved or at least partially corrected. In another embodiment, the system 100 may include instructions executable by the CPU 101 for combining raw data (in the form of gaze direction, and times, brightnesses and patient response to test flashes) and/or corrected post hoc results from successive patient test sessions and analyzing them to render an assessment as to whether or not a patient's condition has progressed. These instructions may include computer readable software components or modules stored in the memory.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Among other things, the following examples illustrate the mapping of the nasal edge of the blind spot in seven normal subjects with a 2 deg grid of test locations, using a custom test station, while gaze direction was monitored with an eye tracker. Records were analyzed to see whether the combined sensitivity and eye movement data could be used to estimate the nature of the blind spot edge. Analysis was carried out for 15 high-variability test locations; the blind spot edge estimates for 12 of these locations were considered to be reasonably consonant with the known general form of the blind spot. One consequence of interpreting the test results using the edge estimates was an average reduction of test-retest variability by 58%. Therefore, among other things, the following examples illustrate that recordings of eye movements during perimetry can be used to generate an improved estimate of scotoma boundaries. An important byproduct of the new estimate is a substantial reduction of test-retest variability.

### Example 1

If a substantial part of test-retest variability results from fixational eye movements, this could be corrected post hoc with eye movement information. 7 normal subjects were tested with a rectangular test array (4×7; 2 deg spacing), extending from 11 deg to 17 deg in the temporal field, encroaching on the blind spot. Additional control locations were placed in nasal, superior, and inferior visual field. Visual stimuli (size III, 0.1 sec) were presented on a CRT (Radius) with a background luminance of 5 cd/m². Testing employed a 2 dB/1 dB two-reversal staircase. Gaze direction was recorded continuously with a video-based eyetracker (ISCAN). Subjects participated in at least two testing sessions.

Subjects showed variability of gaze-direction which could be described by a normal distribution with SD between 0.2 and 0.5 deg. For each subject, one or more test locations showing substantial test-retest variability was selected for further analysis. 15 test locations in 7 eyes were selected. For a given location, gaze direction at the time of each test flash was determined from the eyetracker record. For 5 of the eyes, vertical gaze-direction data were contaminated by lid intrusion; to the extent possible, test locations were selected near vertical blind spot edges for those eyes. Stimuli which elicited “seen” and “not-seen” responses were plotted as functions of gaze-direction, and the data were examined for evidence of a gaze dependence of visual sensitivity. Of the 15 locations analyzed, 3 locations provided clear evidence of gaze-dependence appropriate to the test location. 5 locations were in reasonable accord with an appropriate gaze-dependence. 5 locations did not show a clear dependence. 2 locations produced data that were somewhat contrary to an appropriate dependence.

Thus, the results demonstrate that near steep edges of sensitivity, visual sensitivity is often gaze-dependent, and that eyetracker data could be used to reduce variability. Although some research has failed to find a relationship between test-retest variability and extent of fixational eye movement, as might be expected from the present findings, the power of an uncontrolled variable—test location relative to the sensitivity edge—can account for that.

### Example 2

### Using Gaze-Direction Data to Improve the Estimate of Scotoma Edges

The apparatus employed herein has been previously described in Wyatt H J, Dul M W, Swanson W H. Variability of visual field measurements is correlated with the gradient of sensitivity. Vision Res 2007; 47:925-936. Briefly, visual stimuli were presented on a display monitor (Radius PressView 21 SR, Miro Displays, Inc., Germany) driven by a Power Macintosh G3 computer. The display monitor was a 2100 CRT monitor with 38.0×27.8 cm active area, resolution 832×624 pixels, and frame rate 75 Hz. The monitor was calibrated with a luminance meter (LS-100, Minolta, Japan). The monitor was 75 cm from the recorded eye, so it 29.1 deg horizontally and 21.8 deg vertically at the eye.

Eye movements (gaze direction) and pupil diameter were measured using a PC-based infrared eyetracker (ISCAN EC-101, ISCAN, Inc., Burlington, Mass.) at a sample rate of 60/sec. Experiments were controlled by the Macintosh computer; digital I/O lines connecting the Macintosh and the eyetracker turned eyetracker recording on and off.

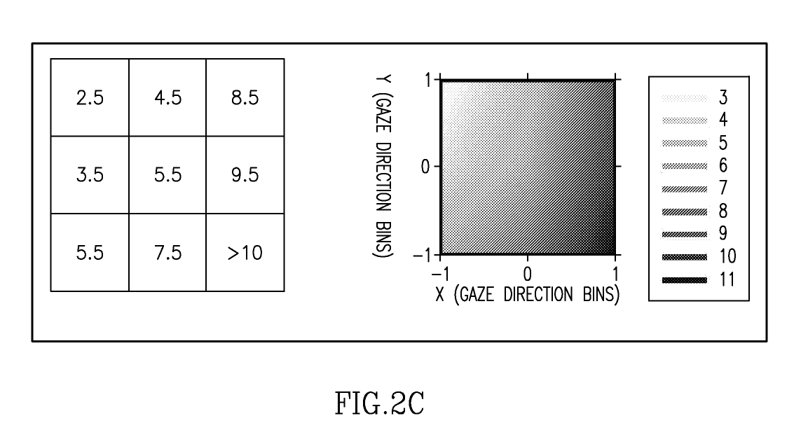

The visual stimuli were Goldmann size III, 100 msec duration, presented on a background luminance of 5 cd/m² at 1 second intervals. The array of test locations is shown in FIG. 3; a rectangular test array (4 wide×7 high, 2 deg spacing) extended from 11 to 17 deg in the temporal visual field, encroaching on the blind spot. Twelve additional test locations were placed in nasal, superior and inferior field, in order to distribute subjects' attention broadly in the visual field. Because the right eye was the recorded eye, the fixation point was located 6 deg left of the monitor center, allowing stimuli out to 20 deg in the temporal visual field. The complete sequence of visual stimuli presented, including location, time, and luminance of each presentation, and subject response (seen/not seen) was recorded by the Matlab program controlling the stimuli.

The stimulus system provided maximum test luminance of 54 cd/m². With the apparatus and parameters employed, this was approximately 16.3 dB (1.63 log units) brighter than threshold for locations away from blind spot at the same eccentricity.

Seven normal subjects participated in these experiments. All subjects had undergone a complete ocular examination within one year of the experiments and had been found to be free of ocular disease. Average age was 30.2 years (range 22-64). Data were collected from the right eye of all subjects. Subjects wore appropriate refractive correction and their left eyes were occluded with an eye patch.

After subjects were set up in the apparatus, the eyetracker was calibrated by having subjects sequentially fixate steady stimuli at an array of 5 locations: the central fixation target, ±3 deg horizontal, and ±3 deg vertical, for 1.5 sec each while the eyetracker recorded “raw” gaze direction data. (Gaze direction data consist of horizontal and vertical coordinates of the location of the pupil center and of the reflection of the eyetracker infrared source in the first corneal surface.) As noted above, the “central” fixation target was actually 6 deg left of the monitor center.

In addition to the x-y calibrations, subjects also participated in a “light-dark” trial, in which they fixated the central calibration target while a large, bright (54 cd/m²) stimulus was turned on and off on the monitor with a 4-second period (2 sec on, 2 sec off, etc), and pupil and gaze direction data were recorded for 16 seconds. This produced substantial pupil responses, and the data were used in analysis of experimental eyetracker data (see below).

The visual field testing, using the test array of FIG. 3, employed a 2 dB/1 dB, two-reversal staircase. Initial stimulus luminance was randomized, starting at 8.09 or 6.23 cd/m². For test locations presented within the average location of the blind spot, initial stimulus luminance was 20.50 cd/m². Subjects pressed a button connected to the Macintosh computer to indicate that they had seen a given test flash. Blank trials were presented at a rate of 1 in 6. The computer generating the test sequences recorded the time, location, and luminance of each test flash and the subject's response.

Gaze direction data were converted from raw data into an estimate of gaze direction by (a) removing blinks using a blink detection algorithm based on rate of change of pupil diameter, (b) calculating the horizontal and vertical distances between pupil center and corneal reflex, (c) correcting the calculated distances according to pupil diameter (see below), and (d) using the calibration values to calculate gaze direction relative to the fixation target. The resulting records of gaze direction as a function of time during the trial were smoothed using 7-bin (7/60 sec=0.117 sec) “boxcar” averaging (running average of 7 bins). Under the conditions of these experiments, five of the seven subjects had pupils large enough and palpebral fissures small enough so that vertical pupil diameter measures were contaminated. Therefore, vertical eye position measurements were unreliable for these subjects and only horizontal eye position data were employed in subsequent analysis.

In previous work, it was shown that pupil centration in the eye is an idiosyncratic function of pupil diameter, but that the behavior is reasonably fixed for a each subject. The “light-dark” trials described above were used to construct functions of pupil center vs. pupil diameter for each subject. Data of the level of step “b” above were then corrected to a standard pupil size, which was taken to be the pupil diameter during gaze direction calibration. This step can be particularly significant in eyes of younger subjects whose pupils can change diameter substantially during field testing. The basis for such pupil changes, which are not a result of changes in environmental illumination, are likely to be responses to changes in balance of parasympathetic/sympathetic activity due to various internal variables (e.g., concern about not doing well on the test, etc.). The effect of pupil changes on video eyetracker data, and a method for compensating for the changes, are described in Wyatt H J. The human pupil and the use of video-based eyetrackers. Vision Res 2010; 50.

For the post hoc analysis, test locations were selected where test-retest variability was high. A “look-up” of data for the staircase information for that location was conducted: for each session, the staircase for the selected test location was extracted from the complete record of the session, and for each test flash presented at the location, the time, luminance and subject response were noted. The time was then used to look up gaze direction at the time of each flash. Pooling data for all sessions for the particular subject created a dataset of actual retinal test location, test luminance and subject response for the selected test location.

The data were then fitted with a spatial function, consisting of two spatial regions of differing sensitivities, separated by a sharp edge. (The use of ramps instead of edges was also assessed, but was generally found to provide little or no improvement compared to edges.) For both regions comprising the spatial function, the probability distribution for seeing a stimulus of contrast z was taken to be Quick's version of a Weibull function (Quick R F, Jr. A vector-magnitude model of contrast detection. Kybernetik 1974; 16:65-7):

$$P ( z ) = 1 - {2}^{[ - {( \frac{z}{α} )}^{β} ]}$$

where α is threshold (P(z=α)=0.5) and β determines the steepness, with larger β giving a steeper curve corresponding to less variability. For 2-dimensional gaze-direction data, there were 6 parameters: angle and placement of the edge, the two sensitivities, α, and β. For 1-dimensional gaze-direction data, the edge was assumed to be vertical, leaving 5 parameters.

The functions were fitted using maximum likelihood estimation (MLE), in which each test delivered is assigned probability P(z) if seen and (1-P(z)) if not seen, the probabilities being evaluated for the current set of parameters. The MLE approach maximizes the product of these probabilities for the entire set of test presentations. The fitting was carried out using a Monte Carlo technique in a program written in IGOR (WaveMetric, Inc.). Some constraints were placed on parameters; in particular, β was allowed to vary from 0.5 to 6 covering a reasonably broad range of steepness. 10⁶ trials were performed for an initial fit and 10⁵ trials were performed to refine the parameters in smaller ranges near the best values from the initial fit.

For each subject, the blind spot contour map of sensitivity generated from the basic test data was fitted by eye with an ellipse, and tangents to that ellipse were used to estimate the orientation (and polarity) of the blind spot edge for each test location studied in the post hoc analysis.

To permit a comparison between test-retest variability with and without consideration of gaze direction, a fit was performed as above, but with the assumption that the eye did not move; the single “fit” value was determined by fitting all of the test contrasts and responses with a single probability function of the type above.

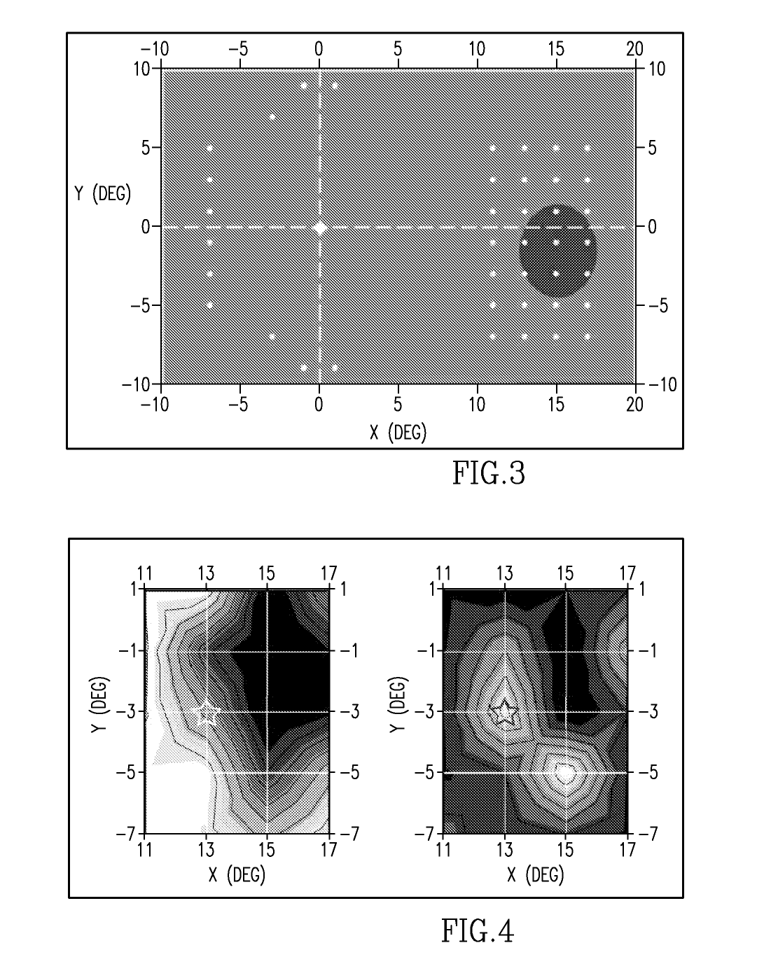

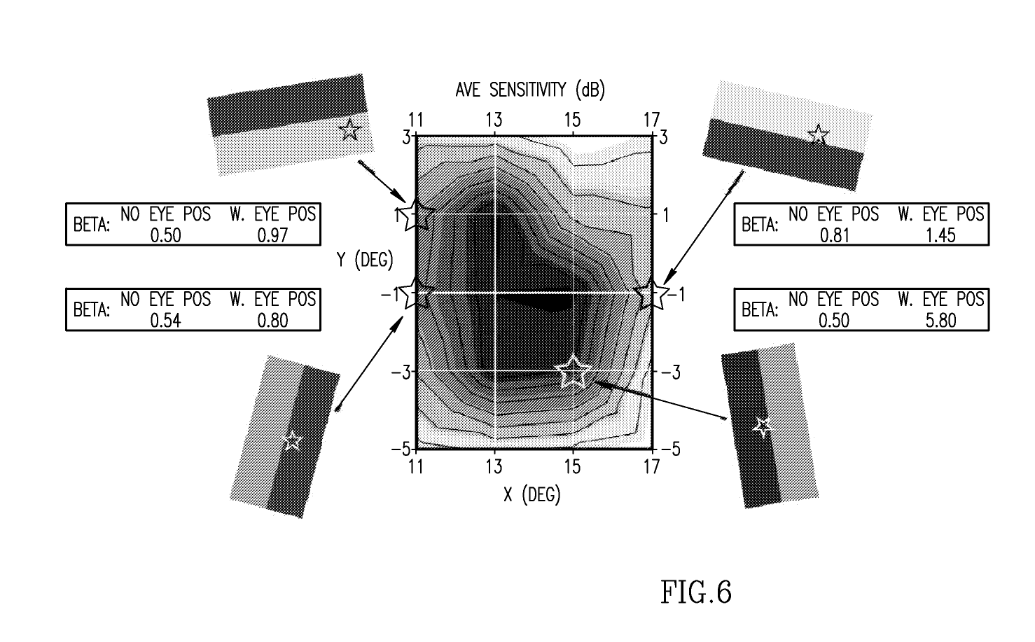

The average sensitivity and test-retest variability for one subject are shown in FIG. 4. (Variability in these plots was taken to be the SD of sensitivity estimates for each test location.) The star indicates the test location selected for post hoc analysis. For the subject of FIGS. 6-7, only horizontal gaze-direction data were available; therefore, the test location selected for subsequent analysis was close to a near-vertical blind spot boundary.

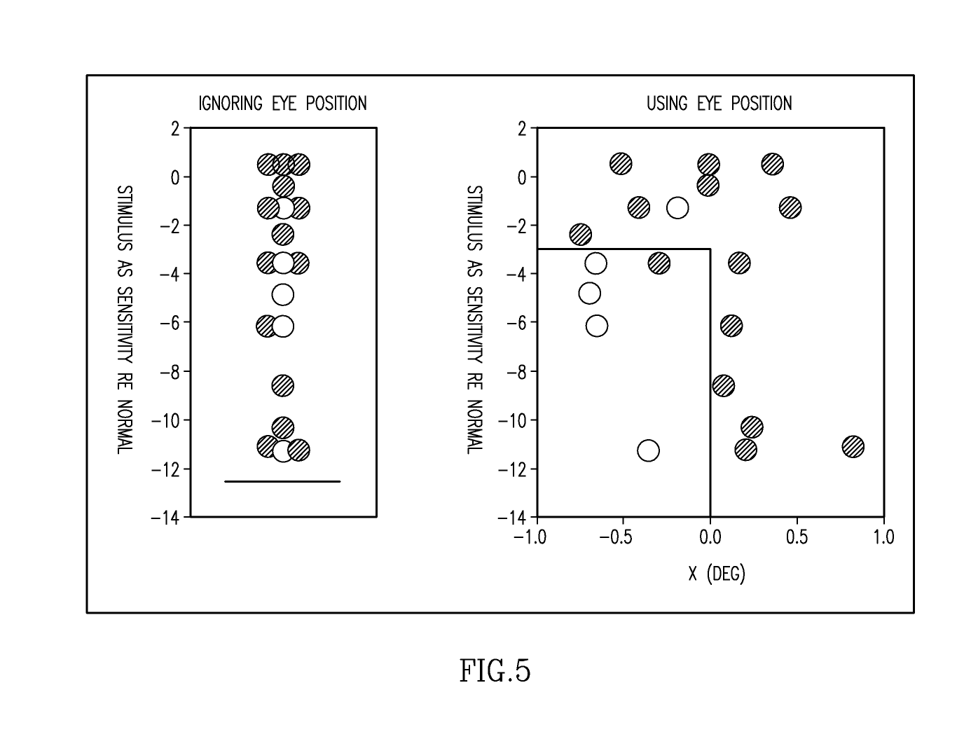

In FIG. 5, the post hoc analysis results for the starred test location of FIG. 4 are shown. The data were fitted well by a step change from normal sensitivity to about −19 dB relative to normal, located 0.3 deg to the right of the nominal test location. Taking gaze direction into account reduced the variability by 81%.

Results for one of the subjects for whom 2-dimensional gaze-direction data were available are shown in FIG. 6. Four test locations were analyzed; the fits for three locations appear plausible, while the fit at (11, 1), although it reduced the variability, was contrary to expectations in terms of the general form of the blind spot (i.e., the fit had greater sensitivity on the side of the edge closer to the blind spot center).

The results for all subjects are summarized in Table 1, in which fits for 2-D data were rated “plausible” if the vector towards the better-seeing side of the edge was within 90 degrees of that estimated by fitting an ellipse to the map of the blind spot for that subject, while fits with 90<difference≦180 were rated “contrary.” For 1-D data (horizontal gaze direction data only), the rating was “plausible” if MLE fit and blindspot map agreed that the worse side was the same (rightward or leftward). The data from S5 proved inadequate to obtain a meaningful fit.

TABLE 1
Summary of fitting results.

| Subject | # locations | plausible fit | contrary fit |
|---|---|---|---|
| S1 | 5 | 2 | 3 |
| S7 | 4 | 4 | |
| S2 | 1 | 1 | |
| S6 | 1 | 1 | |
| S3 | 2 | 2 | |
| S4 | 2 | 2 | |
| S5 | 1 | (inadequate data) | |

The parameter β in the Weibull function is related to steepness of the psychometric function and inversely related to variability. For all test locations, without considering gaze information β was found to be 1.1±1.4 (median 0.8). Taking gaze information into consideration, β was found to be 2.6±2.0 (median 1.9). This amounts to an average reduction of variability of approximately (1.1⁻¹−2.6⁻¹)/1.1⁻¹=58%. Taking gaze information into consideration also increased the log likelihood (in the MLE technique) by 1.6 dB (SD 1.4, median 0.9).

By independently varying the parameters for each fit, it was possible to obtain estimates of the confidence intervals for the key parameters provided by the MLE technique. The 95% confidence interval can be estimated as the width of the parameter range leading to a 1.09 log unit falloff in log likelihood on either side of the maximum (Harvey L. Efficient estimation of sensory thresholds. Behav Res Meth Instrum Comput 1986; 18:623-632). This interval was on average: 0.3 degrees of visual angle (SD 0.3, median 0.3) for placement of the edge; 13 degrees of orientation (SD 25, median 4) for orientation of the edge in cases of 2-D data; and 1.1 (SD 1.4, median 0.8) for the steepness parameter β.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof. Indeed, many variations and alternative elements have been disclosed in embodiments of the present invention, and still further variations and alternate elements will be apparent to one of skill in the art. Various embodiments of the invention can specifically include or exclude any of these variations or elements.

In some embodiments, the numbers expressing quantities of ingredients, properties, conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A method of improving or at least partially correcting test-retest variability in perimetry, comprising:
recording the parameters of gaze direction, time, flash brightness and subject response to a series of test flashes at a series of locations to generate visual field results; and
adjusting test-retest variability in the visual field results by accounting for gaze direction.

2. The method of claim 1, wherein the step of adjusting test-retest variability comprises:
associating the gaze direction with the flash brightness and the patient response for each said time at a said location;
constructing a spatial map of patient responses for each of a series of the said locations; and
accounting for test-retest variability based upon the spatial maps.

3. A method of gauging a progression of a patient's condition, comprising:
in a first session, recording the parameters of gaze direction, time, flash brightness and subject response to a series of test flashes at a series of locations to generate a first set of visual field results;
in at least one additional session, recording the parameters of gaze direction, time, flash brightness and subject response to a series of test flashes at a series of locations to generate an additional set of visual field results for each of the at least one additional session; and
either
(i) adjusting test-retest variability in the first set of visual field results and the at least one additional set of visual field results by accounting for gaze direction, and gauging the progression based on a change in the adjusted first set of visual field results as compared with the adjusted at least one additional set of visual field results; or
(ii) gauging the progression based on a comparison of the parameters recorded in the first session with the parameters recorded in the at least one additional session.

4. The method of claim 3, wherein the step of adjusting test-retest variability in the first set of visual field results and the at least one additional set of visual field results comprises:
associating the gaze direction with the flash brightness and the patient response for each said time at a said location in each of the first set of visual field results and each of the at least one additional set of visual field results;
constructing a spatial map of patient responses for each of a series of the said locations; and
accounting for test-retest variability based upon the spatial maps.

5. The method of claim 3, wherein the patient's condition is selected from the group consisting of glaucoma, other diseases of the eye, and other clinical or disease conditions featuring a deleterious progression in pathology of the eye.

6. The method of claim 3, wherein the patient's condition is glaucoma.

7. A perimetry system, comprising:
a component that interfaces with a patient's eyes to enable performance of visual field testing; and
a computer in electronic communication with the component, and configured to:
record the parameters of gaze direction, time, flash brightness and subject response to a series of test flashes at a series of locations to generate visual field results during the visual field testing,
adjust test-retest variability in the visual field results by accounting for gaze direction, and
generate an output of results to a user of the perimetry system.

8. The perimetry system of claim 7, wherein the computer is further configured to:
associate the gaze direction with the flash brightness and the patient response for each said time at a said location;
construct a spatial map of patient responses for each of a series of the said locations; and
account for test-retest variability based upon the spatial maps.

9. The perimetry system of claim 7, wherein the computer is further configured to:
store the recorded parameters from successive test sessions of the patient; and
gauge a progression of the patient's condition based on a change in either the recorded parameters from the successive test sessions, or a change in the adjusted visual field results from the successive test sessions.

10. The perimetry system of claim 9, wherein the computer is configured to gauge the progression of the patient's condition based on a change in the recorded parameters from the successive test sessions.

11. The perimetry system of claim 9, wherein the computer is configured to gauge the progression of the patient's condition based on a change in the adjusted visual field results from the successive test sessions.

12. The perimetry system of claim 9, wherein the patient's condition is selected from the group consisting of glaucoma, other diseases of the eye, and other clinical or disease conditions featuring a deleterious progression in pathology of the eye.

13. The perimetry system of claim 9, wherein the patient's condition is glaucoma.

14. A computer readable medium having computer executable components for:
recording the parameters of gaze direction, time, flash brightness and subject response to a series of test flashes at a series of locations to generate visual field results during perimetry,
adjusting test-retest variability in the visual field results by accounting for gaze direction, and
generating an output of results.

15. The computer readable medium of claim 14, further comprising components for:
associating the gaze direction with the flash brightness and the patient response for each said time at a said location;
constructing a spatial map of patient responses for each of a series of the said locations; and
accounting for test-retest variability based upon the spatial maps.

16. The computer readable medium of claim 14, further comprising components for:
storing the recorded parameters from successive test sessions of the patient; and
gauging a progression of the patient's condition based on a change in either the recorded parameters from the successive test sessions, or a change in the adjusted visual field results from the successive test sessions.

17. The computer readable medium of claim 16, comprising components for gauging the progression of the patient's condition based on a change in the recorded parameters from the successive test sessions.

18. The computer readable medium of claim 16, comprising components for gauging the progression of the patient's condition based on a change in the adjusted visual field results from the successive test sessions.

19. The computer readable medium of claim 16, wherein the patient's condition is selected from the group consisting of glaucoma, other diseases of the eye, and other clinical or disease conditions featuring a deleterious progression in pathology of the eye.

20. The computer readable medium of claim 16, wherein the patient's condition is glaucoma.
